(19) United States
(12) Patent Application Publication
(10) Pub. No.: US 2025/0345039 A1
(43) Pub. Date: Nov. 13, 2025

(54) APPARATUS AND METHOD FOR DETECTING FLUID-RELATED CONDITIONS IN A PATIENT

(71) Applicant: PULSENMORE LTD., Omer (IL)

(72) Inventor: Elazar Sonnenschein, Omer (IL)

(73) Assignee: PULSENMORE LTD., Omer (IL)

(21) Appl. No.: 18/868,828

(22) PCT Filed: Apr. 20, 2023
(86) PCT No.: PCT/IL2023/050408

§ 371 Date: Nov. 25, 2024

(30) Foreign Application Priority Data
May 29, 2022 (IL) 293427

(51) Int. Cl. A61B 8/00

(52) U.S. Cl. CPC A61B 8/52; A61B 8/4455; A61B 8/461; A61B 8/56

(57) ABSTRACT

A system for detecting fluid-related conditions comprises an ultrasound scanning device adapted to generate ultrasound images at a depth of up to 15 cm from the epidermis, said device being configured such that it can be operated by a non-skilled user.

### FIELD OF THE INVENTION

The present invention relates to the field of ultrasound techniques. More particularly, the invention relates to a device and a method that allow a patient or other non-medically trained person to perform a self-follow-up of the area where an operation has taken place, to detect adverse post-operatorial fluid-related conditions, as well as to identify fluid-related condition resulting from trauma (TFRC).

### BACKGROUND OF THE INVENTION

As a result of surgery, various conditions may occur as fluids accumulate at or near the surgery area, leading to complications. These include, for instance, seromas, hematomas, and abscesses. A seroma is a pocket of clear serous fluid (filtered blood plasma) that may develop in the body after surgery, particularly after breast surgery, abdominal surgery, and reconstructive surgery. A hematoma is localized bleeding outside of blood vessels that may occur after surgery and may involve blood continuing to seep from broken capillaries. A hematoma is initially in liquid form and can spread among the tissues, including in sacs between tissues, where it may coagulate and solidify before blood is reabsorbed into blood vessels. An abscess is a collection of pus that may build up within the body tissue due to various conditions, including after surgery. Signs and symptoms of abscesses include redness, pain, warmth, and swelling.

A variety of surgical procedures may be conductive to post-operatorial, fluid-related (POFR) conditions, such as breast cosmetic surgery, breast reconstruction, and abdominoplasty, a major surgery that removes excess skin and fat from the abdomen. This surgery is common for women who have had many pregnancies or someone who has lost a lot of weight. Seroma formation is thought to occur as plasma from local hemorrhage and other serious fluid accumulates at the site of tissue removal or disruption from surgery or trauma. The fluid collects within scar tissue and can accumulate to a large size causing discomfort and/or can be unsightly. Seromas are commonly seen after abdominal surgery but can be difficult to distinguish from hematomas and hernias based on a physical examination only. On the other hand, in an ultrasound image, seromas are usually anechoic (i.e., seen as black areas) unilocular thin-walled circumscribed fluid collections with posterior through transmission.

One kind of seroma is the abdominal wall seroma, the most common complication after abdominoplasty, one of the most common cosmetic surgeries performed worldwide. Seroma is also the most common local complication associated with abdominoplasty, which increases care costs, reduces patient satisfaction, and has the potential for severe complications for patients. In a systematic review and meta-analysis study reviewing 143 studies (Salari, N., Fatahi, B., Bartina, Y. et ai. The Global Prevalence of Seroma After Abdominoplasty: A Systematic Review and Meta-Analysis. Aesth Past Surg 45, 2821-2836 (2021). https://doi.org/10,1007/s00266-021-02365-6—five studies related to Asia, 55 studies related to Europe, three studies related to Africa, and 80 studies related to the Americas) with a total sample size of 27834 individuals, the global prevalence of seroma after abdominoplasty was obtained as 10.9% (95% Cl: 9.3-3.6.6%) and the highest prevalence of seroma was related to the Europe continent with 12.8% (95% Cl: 10.15-3.9%), occurring most often on postoperative day 11 following abdominoplasty. An ultrasound image of such a seroma is shown in FIG. 1 and indicated by arrow 100. In the paper “Plastic and Reconstructive Surgery”: April 2015—Volume 135—Issue 4—p 691e-698e, Abdominal ultrasounds were performed on postoperative days 4, 11, 18, 25 and 32 on 21 female patients who underwent an abdominoplasty. The researchers identified five abdominal wall regions in which to investigate seroma formation: epigastric, umbilical, hypogastric, right iliac fossa and left iliac fossa. Patients who produced a fluid volume greater than 20 mL throughout the five regions were considered positive for seroma.

To avoid doubts, an ultrasound should be done at the peak of seroma formation (around the 14th postoperative day). If the ultrasound shows that there are more than 20 ml of fluids (as described above), the plastic surgeon should aspirate it to avoid the capsule formation with resulting posterior deformity of the abdomen.

Abdominal wall hematomas may also develop spontaneously, particularly in patients with thrombocytopenia or coagulopathy or those administered systemic anticoagulation medication and most commonly occur in the rectus abdominis muscle (i.e., rectus sheath hematomas). Strong coughing or a significant elevation in blood pressure immediately after surgery may also cause the formation of a surgical site hematoma. Other risk factors include vigorous exercise, straining, vomiting, stress, and alcohol consumption. Small hematomas may resorb after a few days. More severe hematomas that continue to enlarge may require surgery to drain the accumulated blood and/or control any bleeding vessels and reclose the surgical site. A common complication of all hematomas is the risk of infection. Since there is no blood supply to a hematoma—there is a risk of bacteria colonizing the site. Surgical incision dehiscence and delayed healing may also arise if the hematoma is large enough to compress tissues and inhibit oxygen from reaching the surrounding tissue. In an ultrasound image, an acute rectus sheath hematoma appears as a heterogeneously hyperechoic mass or a diffuse enlargement of the rectus muscle without internal Doppler flow. An exemplary hematoma is shown at 200 in FIG. 2. With time, a hematoma may liquefy and become hypoechoic or anechoic, with scattered echogenic components.

An abscess is defined as an infected fluid collection; local symptoms may include edema, hyperemia, fever, gapping wound edges, tenderness, purulent discharge and possibly gangrene (tissue death), and systemic involvement, which may lead to septicemia (spread of the infection into the blood and throughout the body). Additionally, in an ultrasound image, abscesses are typically multilocular complex hypoechoic (not anechoic) collections with thickened septa and hyperemia. An exemplary abscess is shown at 300 in FIG. 3. Gas in an abscess appears in an ultrasound image as echogenic reflectors with ring-down artifacts.

An additional fluid-related condition results from trauma that may happen in various situations, including chest injury by firearm or other means. Focused assessment with sonography in trauma (commonly abbreviated as FAST) is a rapid bedside or field ultrasound examination performed in an emergency by physicians and paramedics as a screening test for blood around the heart (pericardial effusion) or abdominal organs (hemoperitoneum) after trauma. Assuming, for instance, that a soldier was hit by a bullet and there is blood in the chest, the result is a life-threatening situation.

It is thus clear that it is highly desirable to ensure a patient's follow-up after surgery that may involve POFR conditions to avoid complications and irreversible aesthetic and other adverse results. However, this entails onerous activities, such as lengthy follow-up hospitalizations or frequent visits to a medical facility for periodic check-ups.

Similarly, TFRC patients need to be evaluated as close as possible to the time of trauma to determine if life-threatening conditions may exist.

It is a purpose of the present invention to empower patients who have undergone surgery to self-check throughout the desired follow-up period to timely identify situations in which medical intervention is desirable to deal with POFR conditions.

It is another object of the invention to provide a device and procedures that allow a patient to perform such follow-ups.

It is a further object of the invention to allow the quick identification of fluid-related conditions resulting from trauma (TFRC) at any location and without the need to examine the patient in a medical facility.

Other purposes and advantages of the invention will become apparent as the description proceeds.

### SUMMARY OF THE INVENTION

In one aspect, the invention relates to a system for detecting fluid-related conditions, comprising an ultrasound scanning device adapted to generate ultrasound images at a depth of up to 15 cm from the, said device being configured such that it can be operated by a non-skilled user.

The system of the invention is useful in a variety of situations, such as when the fluid-related condition is a post-operatorial condition, such as but not limited to seromas, hematomas, and abscesses.

In one embodiment of the invention, the system comprises a base for an ultrasonic system, the base comprising connection elements adapted to mechanically and electrically connect a smart device to the base and allow the base and the smart device to be moved as a single unit. In some embodiments, the connection elements comprise a cavity adapted to accept and position the smart device.

In embodiments of the invention, electrical power to activate the ultrasonic array is supplied from one of: a rechargeable battery and a DC to DC converter located in the base; a rechargeable battery and a switching power supply, comprised of a power stage and a control circuit, located in the base; and a battery in the smart device (you did not define “smart device”). The system further comprises electronics adapted to operate the ultrasonic array of the ultrasound scanning device and to convey a signal generated thereby to storage elements.

In one embodiment, at least one IMU is integral with the ultrasound scanning device. In another embodiment, at least one IMU is connected to the ultrasound scanning device via a plug-in connection. In a further embodiment, at least one IMU is provided in an element associated with the ultrasound scanning device and moving therewith during operation.

Embodiments of the invention are configured to issue instructions to the system operator that allow scans to be performed by persons not trained for ultrasound scanning, including the patient themselves. The system provides guidance and feedback including alerts re coupling to the skin, contact to skin, gel between transducer and skin, the speed of scan, the organ itself and more.

The scanner comprises an ergonomically-designed housing to be held by an operator and moved across a person's skin. In embodiments of the invention, the housing comprises, or has associated therewith, at least the minimum number of components of the system that must be located on the patient's body to obtain the ultrasound images, such as: i) an ultrasound probe head; ii) electronic components for wired or wireless communication with remote terminals, and iii) a power source.

The system of the invention comprises presets that limit the depth at which ultrasound images are generated to up to 15 cm. This is performed in some embodiments using a transducer which is a 2-12 MHz convex type transducer with ultrasound depth up to 15 cm.

According to some embodiments of the invention, the system comprises software configured to execute at least one of the following: to produce ultrasound images; to analyze the data; to decide which images are of sufficient quality to be displayed on the display screen; to discard low-quality images; to instruct the operator to hold the housing of the scanner in a predetermined manner; to compute the location and attitude of the scanner and, if desired, the roll and pitch yaw; to determine if the scanner is being held such that enough pressure is being exerted on the skin to produce an image of sufficient quality; and to effectively provide instructions how to move the scanner correctly to obtain satisfactory images. Instructions to the operator generated by the software are provided visually on the display screen or audibly from a speaker. In some embodiments, instructions to the operator are provided visually on the display screen or audibly from the speakers by a trained health care professional located at a remote terminal.

Some embodiments of the invention comprise functional settings of the device, selected from one or more of gain compensation, dynamic range, focal length, and depth, which are standardized, thereby preventing the need for the user to make selections.

Also encompassed by the invention is a method for allowing a patient to perform a self-follow-up of the area where an operation has taken place to detect adverse post-operatorial fluid-related conditions, comprising providing said patient with a system of the invention, along with operation instructions therefor. In some embodiments, images generated by the device are relayed to a healthcare professional for evaluation. In other embodiments, an alert is generated if an automated analysis of an image obtained by the device determines that it contains characteristics that may be attributable to a post-operatorial fluid-related condition.

Further encompassed by the invention is a method for performing an emergency focused assessment with sonography in trauma, comprising scanning a patient with a system according to the invention to determine the presence of fluid, such as blood, in the examined area, such as around the heart or around abdominal organs. To be more specific, the fluid absorbs the ultrasonic waves while tissue or bone reflect and absorb only a little of the ultrasonic waves), as can be seen in FIGS. 1-3, the dark area indicates the presence of fluid while the surrounding area has different gray levels. Hence, the dark area suggests that fluid is associated with the image and a specific alert can be presented on the screen. In addition, it is possible to automatically calculate the dark areas by means of pixels and convert them to real measurements of area or volume.

Accordingly, in one embodiment of the invention the system is adapted to generate alerts when an automatic image-processing component detects a dark area that may be indicative of a POFR or TFRC condition. Processing and analysis of the images acquired by the scanner can be processed in both or either the device of the invention and a remote location, e.g., after the images acquired are uploaded to the cloud. Specific embodiments of the invention will handle the automated image analysis differently, when provided, according to the specific requirements of each embodiment.

### BRIEF DESCRIPTION OF THE DRAWINGS

In the drawings:

FIG. 1 shows an exemplary ultrasound scan of an abdominal wall seroma;

FIG. 2 shows an exemplary ultrasound scan of a hematoma;

FIG. 3 shows an exemplary ultrasound scan of an abscess; and

FIG. 4 illustrates an exemplary ultrasound device adapted to carry out the invention.

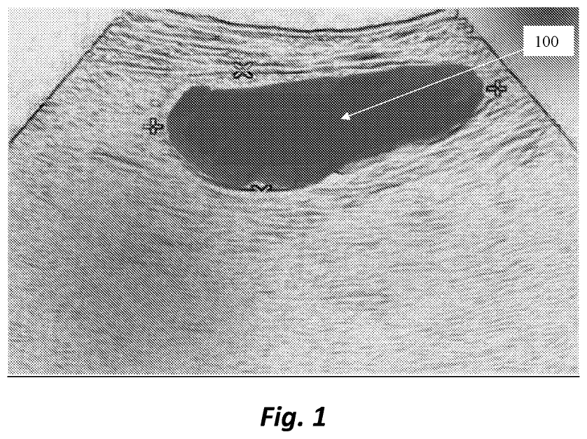

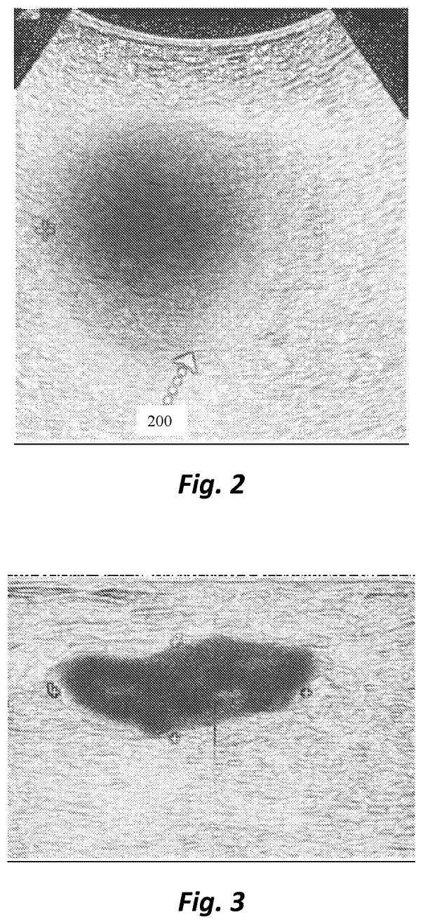

### DETAILED DESCRIPTION OF THE INVENTION

While particular reference will be made herein to abdominal wall seromas, it should be understood that this is for the purpose of illustration and brevity only and that the same description and explanations apply, mutatis mutandis, also to other conditions, such as hematomas and abscesses, as well as to surgery performed in locations other than the abdominal wall, such as for instance, the breasts.

The device for carrying out the invention is an ultrasound device adapted for home use, i.e., an ultrasound device that can be operated by the patient or by untrained individuals (lay users) without the need to perform the operation in a medical environment. One particularly useful such device is the device of U.S. Pat. No. 10,610,194 to the same applicant hereof, the description of which is incorporated herein by reference, which in some embodiments has been modified and accessorized as will be described hereinafter in detail. Other hardware and software elements, whose usefulness in the context of the invention will be appreciated by skilled persons, are described in WO 2021/220263 to the same applicant hereof, the entire description of which is incorporated herein by reference.

In a first aspect, the invention encompasses a system for acquiring ultrasound images of areas affected by surgical operations for detecting POFR conditions and TFRC. The system comprises a scanner adapted to generate ultrasound images at a depth of up to 15 cm from the epidermis of a patient.

While POFR and TFRC conditions can be monitored using conventional ultrasound apparatus operated by skilled medical personnel, for personal use by a patient or other unskilled operator, there is a problem inasmuch as the depth of penetration of the ultrasound radiation is substantial, thus making it difficult for the untrained person to see the area where the POFR or TFRC condition is likely to exist, without being confused by images of other inner body parts. Therefore, in some embodiments, the ultrasound transducer is adapted to perform ultrasound imagining to a limited depth since POFR and TFRC-related conditions to be monitored typically occur at a depth of up to 15 cm. Accordingly, it is convenient for unskilled use that the ultrasound device does not reach internal organs, thus limiting the type of images seen and making it easier for a non-professional individual to monitor the correct body area. An illustrative and non-limitative example of such a device employs a 3.5 MHz convex type transducer with an ultrasound depth of up to 10-15 cm. Of course, it is possible and contemplated by the present invention to use apparatus suitable to perform ultrasound imaging at a depth greater than needed for POFR or TFRC while limiting the imaging depth using the device's setup.

In some embodiments of the system the at least one IMU is one of: a) integral with the scanner; b) connected to the scanner via a plug-in connection; and c) provided in an element associated with the scanner and moving therewith during operation.

Some embodiments of the system are configured to issue instructions to the system operator that allow scans to be performed by persons not trained for ultrasound scanning, including the patient themselves.

In some embodiments of the system, the scans are transmitted to a remote location for analysis by a healthcare professional. In other embodiments of the invention, the detection is performed automatically, either at the patient's end or remotely.

Some embodiments of the system are configured to allow two-way communication between the operator and a remote individual or non-monitored system, wherein the non-monitored system comprises automated image analysis circuitry. The two-way communication can be selected from audio, visual, and video communication and combinations thereof. In some embodiments of the system, two-way video communication is enabled between the operator (which can be the patient themselves, or another person, such as a family member, for example) and the health care professional, enabling them to see each other while the operator is carrying out the scanning procedure to aid the health care professional in interpreting the images and to provide guidance if necessary. In some embodiments, the system is configured such that the system's output is sent directly to a remote healthcare professional and/or to a non-monitored system either in real-time or shortly after the images are acquired.

Embodiments of the system's scanner comprise an ergonomically-designed housing to be held by an operator and moved across a person's skin (who can be the patient or another person). Some embodiments of the housing comprise, or have associated therewith, at least the minimum number of components of the system that must be located on the patient's body to obtain the ultrasound images. In some embodiments of the housing, the minimum number of components in or associated with the housing are: i) an ultrasound probe head; ii) electronic components for wired or wireless communication with remote terminals, and iii) a power source.

In some embodiments of the system, the housing comprises other components that may be arranged in many different configurations in which at least some of them may be located within the housing. In these embodiments, the other components of the system are: v) an Analog Front End (AFE) that transmits and receives ultrasound signals by means of electronic components; vi) a processor containing software; vii) a user interface comprising a display screen and means to accept user's instructions; and viii) at least one memory device to store data and images processed by the software in the processor. In these embodiments, the other components that are not located within the housing are located at a location near the patient but separated from the housing. In these embodiments, the different components that are not located within the housing are in communication with components located within or associated with the housing.

In some embodiments of the system, the electronic components of the AFE comprise transmitters, receivers, amplifiers, and analog to digital (A/D and digital to analog (D/A) converters.

In some embodiments of the system, the software is configured to operate the system and receive and process ultrasound signals received from the AFE to produce ultrasound images and to receive and process inertial measurement signals received from the IMU.

In some embodiments of the system, each of the AFE, IMU, processor, memory devices, and communication components can be provided as separate integrated circuits (ICs) or integrated into one or more ASICs that comprise at least some of the ICs. For the sake of clarity, it should be understood that Processor, for example, might be FPGA or MCU or MCU that includes ADC or DAC. Alternatively, the AFE implemented as ASIC can also include ADC and/or DAC.

Some embodiments of the system comprise additional components. The additional components comprise at least one of: ix) a remote terminal; x) at least one IMU; xi) at least one three-axis magnetometer; xii) at least one pressure sensor; and xiii) a speaker and a microphone for communicating with a remote health care provider.

In some embodiments of the system, all of the other components v)-viii) are contained within a remote terminal connected to the scanner via a wired or wireless communication link. In other embodiments of the system, some of the other components v)-viii) are contained within the scanner, and the remainder located at a remote terminal connected to the scanner via a wired or wireless communication link.

In some embodiments of the system, the remote terminal is a portable communication device. In some embodiments of the system, the portable communication device is a smartphone. In some embodiments of the system, the portable communication device comprises the display, the IMU, and the processor. In some embodiments of the system, the portable communication device fits into a socket in the scanner's housing. In some embodiments of the system, the portable communication device is an integral part of the housing. In some embodiments of the system, the portable communication device is not an integral part of the housing, but is fit into the socket in the housing before performing a scan, moved together with the housing during an ultrasound scan, and, if desired, later detached for other uses. In some embodiments of the system, the portable communication device is connected via a wired or wireless connection to the housing and only the housing is moved.

Illustrative examples of suitable wired communication links include USB, lightning, and fiber optic, but, of course, any additional wired communication is possible. Illustrative examples of wireless communication links include, but are not limited to, Wi-Fi, UWB, Bluetooth, and IR.

The portable communication device can be any of many suitable devices, for example, a mobile phone, tablet, or laptop. Moreover, the housing or a device connected therewith may be in communication with apparatus located in the cloud, adapted to receive data generated by, or associated with, the housing.

In some embodiments of the system, different combinations of one or more IMUs, processing devices and software, memory devices, power sources, and components of the AFE are located either within the housing or in the smartphone. Some embodiments of the system comprise at least one IMU in the smartphone and at least one IMU in the housing.

In some embodiments of the system, the processor is configured to receive data collected by all sensors.

In some embodiments of the system, the software is configured to execute at least one of the following: to produce ultrasound images; to analyze the data; to decide which images are of sufficient quality to be displayed on the display screen; to discard low-quality images; to instruct the operator to hold the housing of the scanner in a predetermined manner; to compute the location and attitude of the scanner; to determine if the scanner is being held such that enough pressure is being exerted on the skin to produce an image of sufficient quality; and to effectively provide instructions how to move the scanner correctly to obtain satisfactory images.

In some embodiments of the system, instructions to the operator that are generated by the software are provided visually on the display screen or audibly from the speakers. In some embodiments of the system, instructions to the operator are provided visually on the display screen or audibly from the speakers by a trained health care professional located at a remote terminal.

In some embodiments of the system, the task of computing the navigation, including the scanner's location, orientation, and time derivatives of them, is carried out by an Inertial Navigation System (INS) comprising a set of three-axis gyroscopes and three-axis accelerometers in the IMU and other sensors; the processor; and software, which is configured to take initial conditions and calibration data and the output from the IMU and other sensors to compute the Navigation, wherein the other sensors can be at least one of a three-axis magnetometer, a pressure sensor, and a camera.

Some embodiments of the system are configured to generate accurate scans of ultrasound signals on the skin and to ensure good value images for diagnostic purposes by using a combination of a pressure sensor and IMU and selecting only images that meet optimal values of the speed of scanning and pressure of the scanner against the skin.

In some embodiments of the system, the INS provides the following types of data:
a. angles of orientation;
b. speed of the scanner; and
c. location of the ultrasound probe head relative to the body's anatomy.

In some embodiments of the system, the speed of the scan is calculated from the angular velocity assuming motion perpendicular to the surface of the body.

In some embodiments of the system, the scan speed is between 1 mm per second and several centimeters per second.

In some embodiments of the system, if the processor determines, during a scan, that not enough pressure is being exerted on the skin, an instruction to increase the pressure is issued to the operator either visually on the display screen, e.g., by displaying a downward pointing arrow, and/or audibly from the speakers. In these embodiments, the processor can determine that not enough pressure is being exerted on the skin by at least one of:
a. analyzing the image and determining that the picture is flat; and
b. measuring the variance of the image's brightness over some region of interest in the image and determining that the variance is smaller than a threshold value.

In some embodiments of the system, the processor contains software configured to determine if an insufficient quantity of water-based gel is interposed between the ultrasound probe head and the skin and to issue an alert to the operator either visually on the display screen and/or audibly from the speakers. In these embodiments, the software can determine if an insufficient quantity of water-based gel is interposed between the ultrasound probe head and the skin by determining if there is a weakening of the signals returning to the probe or weakening of the resulting ultrasound image.

In some embodiments of the system, the processor and software of the system are configured to issue one or more of the following set of instructions to guide an operator to perform a scan:
a. instruct the operator to carry out a calibration procedure if necessary by guiding the operator through the procedure;
b. instruct the operator how to position the patient to take the scan;
c. instruct the operator to position the scanner at a location that will serve as the center of a patient coordinate system;
d. instruct the patient to operate the scanner with the screen facing the patient;
e. provide the operator with instructions, including the direction in which to move the scanner over the surface of the patient's body, how far to move in each direction, the speed with which the scanner should be moved, and the amount of force they should exert to press the scanner against the body;
f. advise the operator that the session is over when enough images of sufficient quality have been collected;
g. to advise the operator, via sound or/and information displayed on a screen, of the conclusion from the scan just performed; and
h. if not done so automatically, advise the operator to forward the images to a health care professional to be interpreted.

In a second aspect, the invention encompasses a method for allowing an operator not trained for ultrasound scanning, be it the patient themself or a second person, to obtain and process ultrasound images of a potential POFR or TFRC condition of a human body. The method comprises:
a. providing a system comprising a scanner adapted to generate ultrasound images at a depth of up to 15 cm from the epidermis; wherein the scanner is the component of the system that is moved by an operator over the surface of a patient's body to obtain the ultrasound images, the system being configured to issue instructions to the operator of the system, that allows scans to be performed;
b. follow the instructions issued by the system.

In an embodiment of the method of the second aspect, the system is the system of the first aspect of the invention.

In an embodiment of the method of the second aspect, the instructions issued by the system are the instructions given by the processor and software of the system of the first aspect of the invention.

In a third aspect, the invention encompasses a method for acquiring ultrasound images of a potential POFR or TFRC condition of a human body. The method comprises providing a scanner adapted to generate ultrasound images at a depth of up to 15 cm from the epidermis, and instructions for an untrained user to operate said scanner.

Some embodiments of the third aspect of the method comprise issuing instructions to the operator of the system that allows scans to be performed by persons not trained for ultrasound scanning, including the patient themselves. Some embodiments of the method of the third aspect comprise transmitting acquired ultrasound images to a remote location for analysis by a healthcare professional. Some embodiments of the method of the third aspect comprise providing circuitry adapted to perform two-way communication between the user and a remote individual or non-monitored system. In some embodiments of the third aspect of the method, the non-monitored system comprises automated image analysis circuitry, and the output of the automated analysis is provided to the user and/or to a healthcare professional. In some embodiments of the third aspect of the method, the two-way communication is selected from audio, visual, and video communication and combinations thereof.

In some embodiments of the third aspect of the method, the system enables two-way video communications between the operator and a health care professional. In some embodiments of the third aspect of the method, the system's output is sent directly to a remote healthcare and/or to a non-monitored system professional in real-time or shortly after images are acquired.

In some embodiments of the third aspect of the method, if the processor determines, during a scan, that not enough pressure is being exerted on the skin, an instruction to increase the pressure is issued to the operator either visually on the display screen, e.g., by displaying a downward pointing arrow, and/or audibly from the speakers. In these embodiments, determining whether not enough pressure is being exerted on the skin can be by at least one of:
a. analyzing the image and determining that the picture is flat; and
b. measuring the variance of the image's brightness over some region of interest in the image and determining that the variance is smaller than a threshold value.

Some embodiments of the third aspect of the method comprise determining through software analysis if an insufficient quantity of water-based gel is interposed between the ultrasound probe head and the skin and issuing an alert to the operator either visually on the display screen and/or audibly from the speakers if an insufficiency of gel is found. In these embodiments of the third aspect of the method, the software can determine if an insufficient quantity of water-based gel is interposed between the ultrasound probe head and the skin by determining if there is a weakening of the signals returning to the probe or weakening of the resulting ultrasound image.

Embodiments of the third aspect of the method comprise guiding an operator to perform a scan by issuing one or more instructions from the following set of instructions:
a. instructing the operator on how to position the patient to take the scan;
b. instructing the operator to position the scanner at a location that will serve as the center of a patient coordinate system;
c. instructing the operator to position the scanner with the screen facing the patient;
d. providing the operator with instructions, including the direction in which to move the scanner over the surface of the patient's body, how far to move in each direction, the speed with which the scanner should be moved, and the amount of force they should exert to press the scanner against the body;
e. advising the operator that the session is over when enough images of sufficient quality have been collected; and
f. if not done so automatically, advising the operator to forward the images to a health care professional to be interpreted.

Hereinafter the invention will be described in detail as a system and method that allow a patient to perform ultrasound scans by themselves. Although conceived as a system for self-use by a person in a home environment, because of its portable nature, the system can also be effectively employed by persons not fully trained for ultrasound scanning, for example, by a family member.

The sensors provided in the device of the invention can include any type of sensor that generates data that is useful in improving and/or adding relevant information to that acquired through ultrasound images. For example, a proximity sensor can be used to alert the user if not enough pressure is applied with the housing to the body, which may result in defective readings.

An additional example of a sensor useful in the context of the invention is an image acquisition element, which can be used, independently of IMU components, to alert the user of coupling problems (e.g., due to insufficient pressure of the device against the body or insufficient gel), or if the user is scanning too fast to generate a good quality image. The abovementioned and other situations that require alerting the user are detected via image processing, which can be performed locally in the housing or remotely by a connected device.

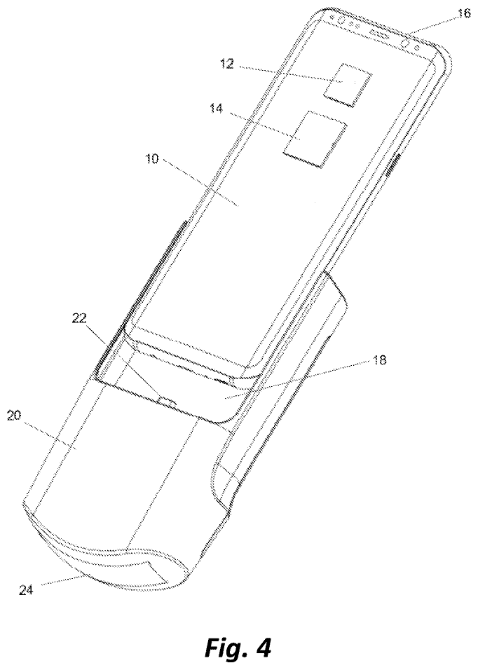

The invention also encompasses a system for obtaining and processing ultrasound images of a potential POFR or TFRC condition of a human body. The system is comprised of many components that be arranged in many different configurations, examples of which will be described herein. The component of the system that is essential to all configurations is called herein a “scanner,” which comprises components of the system that are moved by an operator over the surface of a patient's body to acquire the ultrasound images. The scanner comprises an ergonomically-designed housing to be held by an operator and to be moved across the skin of a person or animal. One illustrative example of such suitable housing, together with a handheld device to be coupled therewith, is shown in FIG. 4. The housing comprises at least the minimum number of components of the system that must be located on the patient's body to obtain the ultrasound images. These elements can be integral with the housing or associated therewith. In the context of this description, the term “associated with” should be interpreted as meaning that the elements or components to which it is referred must not necessarily be integral with the housing but must be in useful cooperation therewith. For instance, where an accelerometer is discussed, it must move together with the housing, and when a communication component is discussed, it must be in communication with any other component located within the housing with which it must exchange data or from which it must receive data. These components are: i) an ultrasound probe head, i.e., an array of ultrasound elements; ii) electronic components for wired or wireless communication with remote terminals, and iii) a power source, e.g., a battery when the system is wireless or power supply in case of a wired system; and in most embodiments iv) optionally, an Inertial Measurement Unit (IMU) comprising the inertial sensors, i.e., a three-axis accelerometer and a three-axis gyroscope, and possibly other sensors, e.g., a three-axis magnetometer and a pressure sensor.

FIG. 4 schematically shows an embodiment in which the display 10, an IMU 12, and the processor 14 are contained in a smartphone 16, which fits into a socket 18 in the housing 20 that contains the other components of the scanner. The smartphone 16 is not necessarily an integral part of the housing 20 but may be fit into the socket 18 before performing a scan, moved as an integral part of the housing 20 during an ultrasound scan, and later detached for other uses. The smartphone 16 is electrically connected to the housing 20 by a connector 22 in the socket 18, which fits into a standard port on the smartphone 16. Seen in FIG. 4 is ultrasound probe head 24 at the bottom of housing 20. The term “smartphone,” as used herein, refers to any portable communication device for which a fitting seat can be created in a housing, such as housing 20 of FIG. 4, and is not intended to limit the invention to any particular type of communication device, existing or to be developed. The smartphone was chosen in this example to illustrate the invention only since it is a widespread device available to most people.

In another embodiment, the smartphone is connected via a cable or a wireless connection to the housing and only the housing or the probe itself is moved, i.e., the smartphone does not necessarily have to move in unison with the ultrasound probe.

In other embodiments, different combinations of processing devices and software, memory devices, power sources, other optional components such as IMUs, and components of the AFE, are located either within the housing or in the smartphone.

In some embodiments of the invention that comprise one or more IMUs, the inertial sensors are not integral with the housing. Instead, the inertial sensors of the smartphone or the like portable device (that will be discussed later) can be used, or add-on inertial sensors can be connected to the housing prior to use. In another embodiment of the invention, the housing can be a “docking housing,” i.e., a housing that only comprises components essential for connecting functional components such as sensors of various types thereto, and said sensors can be connected to the docking housing as needed. This embodiment allows selecting appropriate types of sensors for a given use, which can be added as “plug and play” components to the housing.

Other typical components of the system are: v) an Analog Front End (AFE) that transmits and receives ultrasound signals by means of electronic components including, inter alia, transmitters (pulsers), receivers, amplifiers, and analog to digital (A/D and digital to analog (D/A) converters; vi) a processor containing software configured to operate the system and to receive and process ultrasound signals received from the AFE to produce ultrasound images and to receive and process inertial measurement signals received from the IMU, when used; vii) a user interface comprising a display screen and means to accept user's instructions, e.g., a keyboard or touch screen; and viii) a memory device or devices to store data and images processed by the software in the processor. In different embodiments, some or all of these components may be located within the scanner's housing or at a location near the patient but separated from the housing. There are many options for arranging these components, which will be easily appreciated by the skilled person.

The electronic components, i.e., the AFE, processor, memory devices, and communication components, can be provided as separate integrated circuits (ICs) or integrated into one more ASICs that comprise all or some of the ICs.

Optional components of the system include: ix) a remote terminal, e.g., a smartphone, tablet, PC, or similar communication and computing device that is located near the operator or far from the operator, e.g., in a clinic or doctor's office; x) one or more IMUs; x) at least one three-axis magnetometer; xi) at least one pressure sensor; and xi) a speaker and microphone for communicating with a remote health care provider.

In some embodiments of the system, all the components v)-viii) are contained within (or on in the case of the display) the scanner's housing.

In some embodiments of the system, all the components v)-viii) are contained within a remote terminal connected to the scanner via a wired or wireless communication link; wherein the wireless link can be formed using any known technology, e.g., Cellular, WIFI or Bluetooth.

In some embodiments of the system, some of the components v)-viii), e.g., some or all of the components of the AFE, are contained within the scanner and the remainder in the remote terminal, which is connected to the scanner via a wired or wireless communication link.

The processor is configured to receive data collected by all sensors and contains software that is configured, inter alia, to produce ultrasound images; to analyze the data; and, in some embodiments, to decide which images are of sufficient quality to be displayed on the display screen; to compute the location and attitude of the scanner, to discard low-quality images; to instruct the operator to hold the housing of the scanner in a predetermined manner, e.g., such that the display screen (or a designated symbol on the housing surface in embodiments in which the display is remotely located) always faces her/him; to determine if the scanner is being held such that enough pressure is being exerted on the skin to produce an image of sufficient quality; and to effectively provide instructions how to move the scanner correctly in order to obtain satisfactory images by means of an intuitive graphical cue presented on the display screen. In other embodiments, the instructions to the operator are provided visually or audibly on the display screen and speakers or by a trained health care professional located at a remote terminal.

Instructions that may be delivered to the operator via the smartphone or other communication element in the basis may include, for example:
The screen is not facing you—please keep it perpendicular to your body;
The image is not clear—please apply more pressure or add more gel;
You're moving too fast—please slow down; and
Please move the housing to the right.

According to embodiments of the invention, the task of computing the scanner's location, orientation, and time derivatives of them is carried out by an Inertial Navigation System (INS). The INS is comprised of the IMU, i.e., a set of three-axis gyroscopes and three-axis accelerometers and other sensors, e.g., usually a three-axis magnetometer and a pressure sensor; the processor; and software configured to take initial conditions and calibration data and the output from the IMU and other sensors to compute the Navigation.

It is also possible to use other sensors in addition to the IMU, magnetometer, and pressure sensor to improve accuracy. For example, in a mobile phone, a front camera points toward the user and a rear camera points toward objects in the room. For the embodiment in which a smartphone, which fits into a socket in the housing that contains the other components of the scanner, at the beginning of the scan, the rear camera points towards a particular object in the room. During the scan, the rear camera moves with the housing, and the movement relative to the object in the image can be tracked using an optical flow method, thereby providing another piece of information to the navigation algorithm that can be used to correct errors.

As will be appreciated by the skilled person, using a probe and focusing on a depth that is up to 15 cm, as done according to embodiments of the invention, will cause a loss of the details of the layers of the skin. This causes no concern since what is needed is to focus on fluid collections that are deeper than superficial skin layers. As will be apparent to the skilled person, different probes shapes (convex, linear, micro convex, phase array) can be used with a combination of frequencies to operate at any depth.

In embodiments of the invention, the system can be configured to generate accurate scans of ultrasound signals on the skin and to ensure good value images for diagnostic purposes by using a combination of a pressure sensor and IMU and selecting only images that meet optimal values of the speed of scanning and pressure of the scanner against the skin.

These sensors of the inertial measurement unit (IMU) or inertial navigation system (INS) can be implemented using a single chip ASIC that contains all or some of them or as a discrete chip that implements each sensor separately or as combinations of sensors.

The IMU provides several types of data:
1. Angles of orientation, which are used to:
   a) Provide the user with instructions on how to hold the scanner to get the best images, b) Provide a physician or other professional with the continuous orientation of the probe at the time a scan was taken in order to facilitate interpretation of the image. This information can be presented as an overlay on the ultrasound image.
2. Speed of the scanner, which is used to:
   a) Provide the user with instructions on how to move the scanner to get the best images. This information can be provided to a remotely located physician so they may be aware of how the scan is performed with all the alerts that the operator experienced.
   b) Filter out images that are unlikely to contain useful information. For example, the criteria for deleting images could be a speed greater than 10 cm/sec or could also be 1 cm/second in a situation where slow scanning is required to detect a specific phenomenon—for example, self-scanning of the inferior vena cava (IVC) in the case of congestive heart failure (CHF) patients.
3. Location of the ultrasound probe head relative to the body's anatomy, which is used to:
   a) provide the user with instructions on how to scan the whole area of interest to fully cover the organ of interest;
   b) provide a physician or other professional with the scanner's continuous orientation at the time a scan was taken in order to facilitate interpretation of the image.

The IMU, like other devices, is not perfect. IMU errors, upon integration, form drift, an error that increases over time, and therefore, the error of the computed location and orientation quickly propagates over time. An example can best illustrate the problem. Assume that due to measurement noise and other imperfections, the device's orientation is known with an error of one milli-radian. This error is considered very small given the quality of, for example, a typical smartphone's IMU. Given this error, the processor misinterprets the accelerometer readings and interprets the projection of the gravitation as a horizontal acceleration of approximately one cm/sec². This small acceleration error results in a location error of 18 meters over one minute, clearly well beyond the acceptable error. Thus, the processor must have some additional information and must assume some restrictions in order to provide meaningful navigation.

The IMU installed in smartphones nowadays is based on Micro-Electro-Mechanical Systems (MEMS) technology. MEMS technology provides tiny, efficient, affordable sensors but suffers from inherent imperfections resulting in measurement errors. The errors can be divided into biases and noise. Formally, the only difference is that a bias varies slowly whereas noise varies quickly. However, over the time period relevant to ultrasound scans and for illustrating the problem, biases can be regarded as constant, and noise can be regarded as absolutely random.

Thus, due to biases, the IMU of a motionless device still produces measurements as if the device is rotating and accelerating. In order to calibrate the IMU and find the biases, a calibration procedure must be presented. Still, no calibration is perfect due to noise, and some residual bias always remains. Also, noise, albeit random, only sums up to zero after an infinite number of measurements. In practice, the expected value of the noise is the square root of the number of measurements times the standard deviation of the noise.

As said, the IMUs installed in smartphones so far are all MEMS-based, subject to strict limits of cost, size and energy consumption, and therefore, are very similar to each other. Their noise and bias figures are, in principle, the same.

As a result of biases and noise, and given the quality of MEMS IMUs, the navigation process must integrate more measurements and utilize some prior assumptions in order to mitigate the IMU errors. When scanning with the scanner, the distances moved are small, and the scanning speed is relatively slow, which frequently results in the noise generated in the IMU being larger than the signal. Typical distances for these scans are in the range of several millimeters and up to several tens of centimeters and typical speeds of 1 mm/sec to several centimeters per second. Thus, successful navigation relies on optimal calibration allowed by the system, the mission, the user, and the integration of other available cues.

Some bias errors are calibrated at the manufacturing level. However, some biases vary over time and must be calibrated before use, as described in detail in WO 2021/220263, to which reference is made.

The ultrasound scan depends on holding the scanner such that some pressure is exerted on the skin. When the pressure drops, the scanner produces a flat image. The processor analyzes the image and, upon concluding that the picture is flat or using similar criteria such as measuring the variance of the image's brightness over some region of interest instead of the entire picture. If the brightness is smaller than a threshold value, it issues an instruction to the operator to increase pressure. In an embodiment, this instruction may include, as an example, the appearance of a down-pointing arrow on the display screen with vocal instruction to increase pressure on the skin.

It is common to use a water-based gel to provide a smooth media for the ultrasound beams to propagate from the probe to the body; otherwise, the beams will be attenuated when passing through the air. Using the resulting signal or image, it is possible to determine whether the coupling between the probe and the body is sufficient. This, for example, can be determined by the weakening of the signals returning to the probe or by the weakening of the resulting ultrasound image.

The speed of the scan can be calculated from the angular velocity. The processor assumes motion perpendicular to the surface of the body. The range of permitted velocities is a characteristic of the scanner and is typically several centimeters per second. This slow motion produces a radial acceleration of as little as one millimeter per second squared, which means that the acceleration of gravity can be used by the EKF as a good approximation of the acceleration in the downward direction. Thus, when the computed velocity is not within a permitted range, the scan is discarded, and an instruction is issued to the patient to go slower.

Combining the speed and orientation, the scanner can ensure that the user is instructed to cover a predetermined range of angles and do it within the permitted velocity range. Adding the quality of the image produced by the image processing, proper pressure on the skin is also maintained. Altogether this ensures a good examination.

The scanner is a “black box” as far as the operators of the scanner are concerned. The patients (or other operators) only have to follow the visual or audible instructions that they receive from the components of the system or from a sonographer in the case of Telemedicine. It is also possible to show video instructions by means of animations.

### Example 1: Coupling Alert

The following exemplifies a coupling alerting procedure according to one particular embodiment of the invention. The procedure involves the following steps:
a. Image acquisition—Construction of the ultrasound image from the echoes received from the body organs to the transducer.
b. Image pre-processing—At the beginning of the process, the frames undergo image pre-processing that normalizes the variance between frames from different scans.
c. Total Black Frame (TBF) test—following the image pre-processing, the algorithm performs a TBF test. In the TBF test, the percentage of pixels that are absolute black in the entire current frame are examined in order to find frames that qualify for a TBF condition.
d. Coupling condition classification—The coupling condition of any side (left/right) of each frame is made by a decision tree classifier.
e. Buffer test—Each classification is saved in a buffer on a length of 16 decisions. If 80% of the decisions indicate an insufficient coupling, the user is instructed to improve skin contact or add more gel.
f. Displays an alert to the operator—While performing a scan, the user receives real-time feedback regarding the coupling condition. In the case of 80% frames with insufficient coupling, the user is instructed to improve skin contact or add more gel.
g. Add image to recording—If good coupling is detected, the frame is recorded.
h. Displays an alert to the operator (TBF)—If no coupling is identified, the system guides the user to hold the cradle tighter to the skin.
i. Drop image from recording—in TBF cases, the frame is not kept, thus improving the received image.
j. Display image on screen—all images are displayed on the screen (TBF, insufficient coupling and good coupling).

### Example 2: “Scan Too Fast” Alert

The following illustrates a procedure for dealing with a user who is moving the housing too fast to produce a good quality scan.

From the scanned image, the following two steps are performed to acquire a value for the scan speed:
a. Detecting a change in a large portion of the image; and
b. Detecting the optical flow to obtain the speed.

To estimate the change, a temporal standard deviation is calculated over 6 frames. If a significant change is detected in more than 0.5% of the total scan pixels, this signifies that a movement has been made.

To evaluate an overall change in the picture, a change per second in pixel intensity is evaluated across the image. A pixel temporal standard deviation is used as an estimator for change. For an image I(x, y, n) where “n” is the number of frames, and each frame is taken in time t(n). The following calculation is used to evaluate change:

$$σ ( x , y , n ) = \frac{1}{6} \sqrt{{\sum }_{i = n - 5}^{n} {I ( x , y , i )}^{2} - {( {\sum }_{i = n - 5}^{n} I ( x , y , i ) )}^{2}}$$

This yields a measurement of the amount of change per frame. To evaluate the change in time, the value is normalized by the mean FPS

$${σ}^{*} ( x , y , n ) = σ ( x , y , n ) \cdot \frac{6}{{\sum }_{i = n - 5}^{n} t ( n ) - t ( n - 1 )}$$

In the next step, the number of pixels that have changed dramatically is calculated:

$$C ( x , y , n ) = \{ \begin{matrix}1 ; & {σ}^{*} > Th \\ 0 ; & else\end{matrix}$$

where Th is a threshold empirically selected to distinguish between noise- and motion-related changes. To determine the required threshold value, in one embodiment, the mean standard deviation between 10 sequential frames is calculated for 100 scans, which have been taken with the ultrasound device of the invention. It should be taken when the device is held still. Th value are calculated as the mean and three standard deviations of the calculated values.

Now the sum of C is calculated to understand which percent of the image has changed.

If

$$\frac{{\sum }_{x = 1}^{X} {\sum }_{y = 1}^{Y} C ( x , y , n )}{XY} > 0.5 ,$$

the frame is considered as a moving frame.

For a moving frame, an optical flow Vₓ, V_{y} is calculated using the Lucas-Kanade method with pyramids. Corners in the center of the image are used for calculation using Harris corner detector.

The optical flow gives the speed per frame. In order to attain the speed in time, it should be normalized by the FPS.

$$[ {V}_{x}^{*} , {V}_{y}^{*} ] = \frac{[ {V}_{x} , {V}_{y} ]}{t [ n ] - t [ n - 1 ]}$$

It should be understood that since the depth of the scanning is smaller, in embodiments of the invention, the functional settings of the device (such as gain compensation, dynamic range, focal length, depth etc.) are standardized so that the user does not have to make any selections. In such embodiments, the user only needs to turn on the device and perform the scan on the relevant body area, and the device will automatically generate images of the POFR or TFRC area. In some embodiments, using an ultrasound transducer adapted to image internal organs as well, presets are provided in the device to allow the user to select the appropriate setup. For instance, setups for “POFR” or “Fetus” can be selected by the user according to the device's intended use.

Although embodiments of the invention have been described by way of illustration, it will be understood that the invention may be carried out with many variations, modifications, and adaptations without exceeding the scope of the claims.

What is claimed is:

1. A system for detecting fluid-related conditions, comprising an ultrasound scanning device adapted to generate ultrasound images at a depth of up to 15 cm from the epidermis, said device being configured such that it can be operated by a non-skilled user.

2. A system according to claim 1, wherein the fluid-related condition is a post-operatorial condition.

3. (canceled)

4. A system according to claim 1, comprising a base for an ultrasonic system, the base comprising connection elements adapted to mechanically and electrically connect a smart device to the base and to allow the base and the smart device to be moved as a single unit.

5. A system according to claim 4, wherein the connection elements comprise a cavity adapted to accept and position the smart device.

6. A system according to claim 4, wherein electrical power to activate the ultrasonic array is supplied from one of: a rechargeable battery and a DC to DC converter located in the base; a rechargeable battery and a switching power supply, comprised of a power stage and a control circuit, located in the base; and a battery in the smart device.

7. (canceled)

8. A system according to claim 1, comprising at least one IMU integral with the ultrasound scanning device or connected to the ultrasound scanning device via a plug-in connection.

9. (canceled)

10. (canceled)

11. The system of claim 1, which is configured to issue instructions to the operator of the system that allow scans to be performed by persons not trained for ultrasound scanning, including the patient themselves.

12. The system of claim 1, wherein the scanner comprises a housing that is ergonomically designed to be held by an operator and moved across the skin of a person, wherein the housing comprises, or has associated therewith, at least one minimum number of components of the system that must be located on the patient's body to obtain the ultrasound images, and wherein the minimum number of components in, or associated with the housing are: i) an ultrasound probe head; ii) electronic components for wired or wireless communication with remote terminals, and ii) a power source.

13. (canceled)

14. (canceled)

15. (canceled)

16. The system of claim 12, which comprises presets that limit the depth at which ultrasound images are generated to up to 15 cm.

17. The system of claim 1, wherein the ultrasound scanning device comprises a transducer which is a 2-6 MHz convex type transducer with ultrasound depth up to 15 cm.

18. The system of claim 1 comprising software configured to execute at least one of the following: to produce ultrasound images; to analyze the data; to decide which images are of sufficient quality to be displayed on the display screen; to discard low-quality images; to instruct the operator to hold the housing of the scanner in a predetermined manner; to compute the location and attitude of the scanner and, if desired, the roll and pitch yaw; to determine if the scanner is being held such that enough pressure is being exerted on the skin to produce an image of sufficient quality; and to effectively provide instructions how to move the scanner correctly in order to obtain satisfactory images.

19. The system of claim 18, wherein instructions to the operator that are generated by the software or by a trained health care professional located at a remote terminal and are provided visually on the display screen or audibly from a speaker.

20. (canceled)

21. The system of claim 1, wherein the functional settings of the device, selected from one or more of gain compensation, dynamic range, focal length, and depth, are standardized, thereby preventing the need for the user to make selections.

22. A method for allowing an operator not trained for ultrasound scanning, be it the patient themself or a second person, to obtain and process ultrasound images of a potential POFR or TFRC condition of a human body, comprising:
a. providing a system comprising a scanner adapted to generate ultrasound images at a depth of up to 15 cm from the epidermis; wherein the scanner is the component of the system that is moved by an operator over the surface of a patient's body to obtain the ultrasound images, the system being configured to issue instructions to the operator of the system, that allows scans to be performed;
b. following the instructions issued by the system.

23. (canceled)

24. The method of claim 22, wherein images generated by the device of claim 1 are relayed to a healthcare professional for evaluation.

25. The method of claim 22, wherein an alert is generated if an automated analysis of an image obtained by the device of claim 1 determines that it contains characteristics that may be attributable to a post-operatorial fluid-related condition.

26. (canceled)

27. A method for performing emergency focused assessment with sonography in trauma, comprising scanning a patient with a system according to claim 1, to determine the presence of fluid in the examined area.

28. (canceled)

29. The method of claim 25, wherein the examined area is around the heart or around abdominal organs.

30. The method of claim 22, wherein the system is adapted to generate alerts when an automatic image-processing component detects a dark area that may be indicative of a POFR or TFRC condition.

31. (canceled)
